(19) United States
(12) Patent Application Publication
(10) Pub. No.: US 2004/0010318 A1
(43) Pub. Date: Jan. 15, 2004

(54) Conformable endplates for artificial disc replacement (ADR) devices and other applications

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave. Suite 400, Birmingham, MI, 48009-5394, US

(21) Appl. No.: 10/438,604

(22) Filed: May 15, 2003

Related U.S. Application Data
(63) Continuation-in-part of application No. 10/421,435, filed on Apr. 23, 2003.
(60) Provisional application No. 60/380,631, filed on May 15, 2002.

(51) Int. Cl. A61F 2/44

(52) U.S. Cl. 623/17.16; 623/17.11

(57) ABSTRACT

An anatomical artificial disc replacement (ADR) device includes a tray having a surface which is convex to better conform to a concavity in a vertebral endplate. In different preferred embodiments, the tray may be constructed of multiple pieces adapted to conform to the vertebral endplate; a flexible material such as a malleable metal to fit the vertebral endplate; or a substrate and an attachable convex piece configured to conform to the concavity. Alternatively, the tray includes a substrate and an injectable material that hardens in situ to fill the concavity. The injectable material may be a liquid metal or a polymer, and may be injected along diverging or converging paths to minimize pull-out.

### REFERENCE TO RELATED APPLICATION

This application claims priority from U.S. Provisional Patent Application Serial No. 60/380,631, filed May 15, 2002; and is a continuation-in-part of U.S. patent application Ser. No. 10/421,435, filed Apr. 23, 2003. The entire contents of both applications are incorporated herein by reference.

### FIELD OF THE INVENTION

This invention relates generally to artificial disc replacement and, in particular, to conformable endplates for ADR devices.

### BACKGROUND OF THE INVENTION

Premature or accelerated intervertebral disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, replace either the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

Total disc replacement (TDR) devices conventionally cover the vertebral endplates with metal trays or plates. Generally, the trays are flat. The vertebral endplates are rarely flat. The inferior vertebral endplate, in particular, has a concavity which is usually centered in the posterior portion of the vertebrae. FIG. 1 illustrates normal anatomy. The depth of the concavity 102 and the center of the concavity vary from patient to patient. The superior endplate of vertebrae may also have a concavity. The concavity on the superior surface of vertebrae is usually shallower than the concavity on the inferior surface of vertebrae.

The endplates of vertebrae must be shaped to fit flat TDR trays. Shaping involves cutting or shaving the “high” parts of the endplate to a point level with the base of the concavity. Generally, the periphery of the endplate is removed. At times, a generous amount of vertebra must be removed to support the entire tray. Unfortunately, most of the support for the tray comes from the stronger bone around the periphery of the endplate. Thus, excessive endplate removal weakens the support for the tray.

TDR trays with convex surfaces have been proposed as an alternative to cutting the vertebral endplates. However, matching the convexity of a TDR tray with the wide variety of endplate concavities would require a prohibitively large inventory.

### SUMMARY OF THE INVENTION

This invention resides in an anatomical artificial disc replacement (ADR) device comprising a tray having a surface which is convex to better conform to a concavity in a vertebral endplate.

In different preferred embodiments, the tray may be constructed of multiple pieces adapted to conform to the vertebral endplate; a flexible material such as a malleable metal to fit the vertebral endplate; or a substrate and an attachable convex piece configured to conform to the concavity.

Alternatively, the tray includes a substrate and an injectable material that hardens in situ to fill the concavity. The injectable material may be a liquid metal or a polymer, and may be injected along diverging or converging paths to minimize pull-out.

### BRIEF DESCRIPTION OF THE DRAWINGS

FIG. 1 illustrates the normal anatomy;

FIG. 2A illustrates a preferred embodiment of the present invention;

FIG. 2B shows the configuration of FIG. 2A from a side-view perspective;

FIG. 3A shows a further preferred embodiment of the present invention;

FIG. 3B shows the configuration of FIG. 3A from a side-view perspective;

FIG. 4A show a further alternative embodiment which places removable hemi-convex pieces over a portion of the tray;

FIG. 4B shows the configuration of FIG. 4A from a side-view perspective;

FIG. 5A is a lateral view of an alternative embodiment of the invention;

FIG. 5B shows a sagittal cross section of a further embodiment of the present invention;

FIG. 6 is a coronal cross section of the tibia; and

FIG. 7 is a coronal cross section of the tibia and a further embodiment of the present invention.

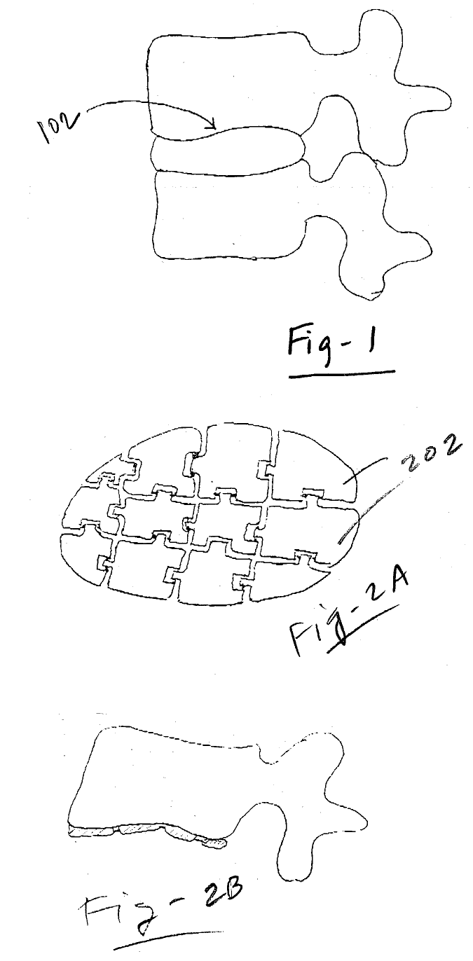

### DETAILED DESCRIPTION OF THE INVENTION

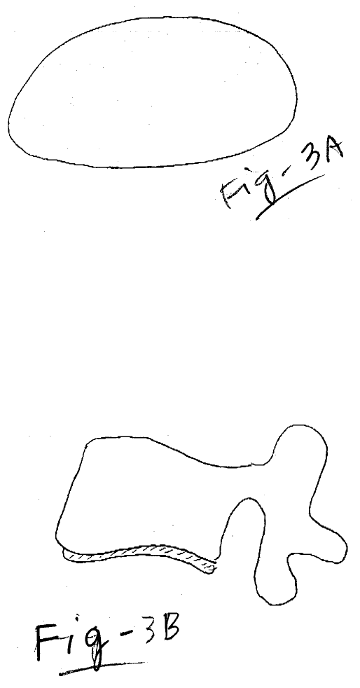

This invention improves upon the prior art by providing TDR trays that can change shape or be adapted to fit the vertebral endplate. FIG. 2A illustrates a preferred embodiment of the invention, wherein the endplate is constructed from interlocking pieces 202, which may themselves be malleable for even greater conformity. FIG. 2B shows the configuration of FIG. 2A from a side-view perspective.

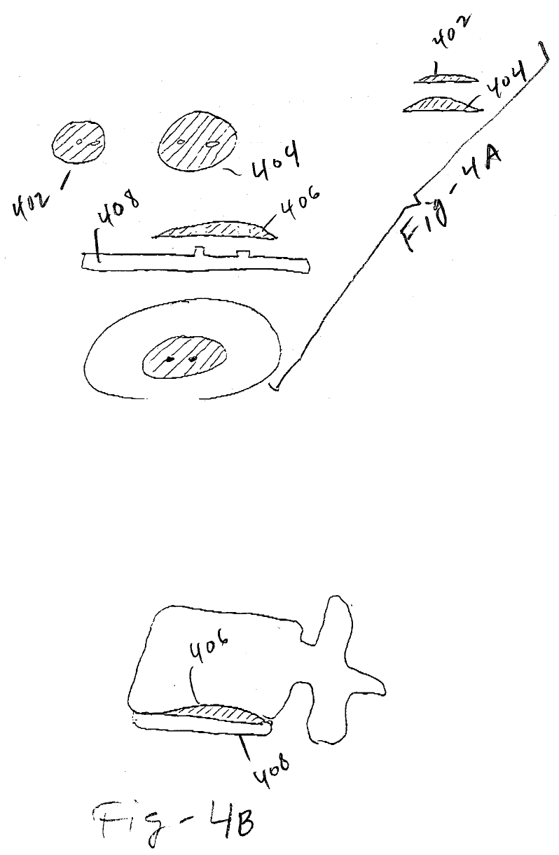

In another preferred embodiment, the trays are continuous but flexible or malleable to fit the vertebral endplate, as shown in FIG. 3A. FIG. 3B shows the configuration of FIG. 3A from a side-view perspective.

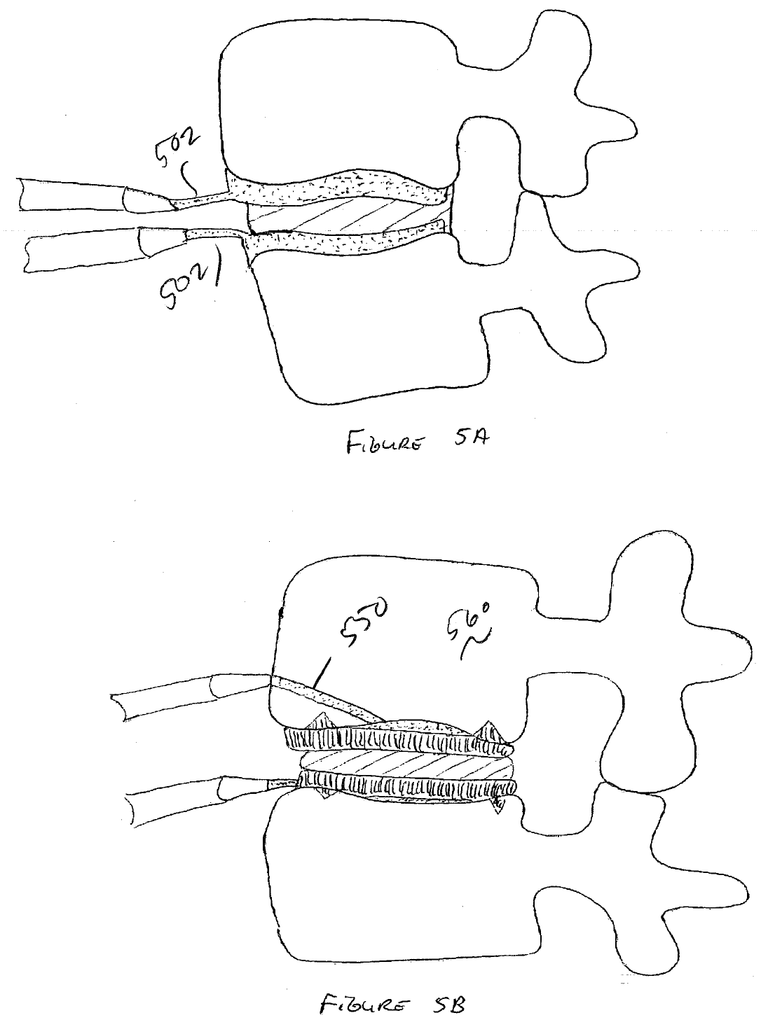

An alternative embodiment places removable hemi-convex pieces 402, 404, 406 over a portion of the tray 408, as shown in FIG. 4A. FIG. 4B shows the configuration of FIG. 4A from a side-view perspective.

As opposed to rigid or semi-rigid pieces, the invention also anticipates the use of materials that harden in-situ. The liquid form of certain metals, for example, could be used to “customize” an ADR EP to the surface of a vertebral endplates. Customization would improve surface contact to prevent excessive loading of the vertebral EPs. Customization could also be used to improve attachment of the ADR EP to the vertebral EPs. Use of in-situ curing polymers, including PMMA, could also be used according to the invention to form “custom” ADR EPs.

Overall, the invention is directed to both fully formed in-situ embodiments and partially formed in-situ embodiments. Fully formed embodiments use a mold to make the entire ADR EP. Partially formed adds in-situ hardening materials to a standard partially formed ADR EP, to “customize” the ADR EP. Method aspects of the invention also include pressurizing conformable ADR EPs to shape them and to press fit them into the vertebral EPs.

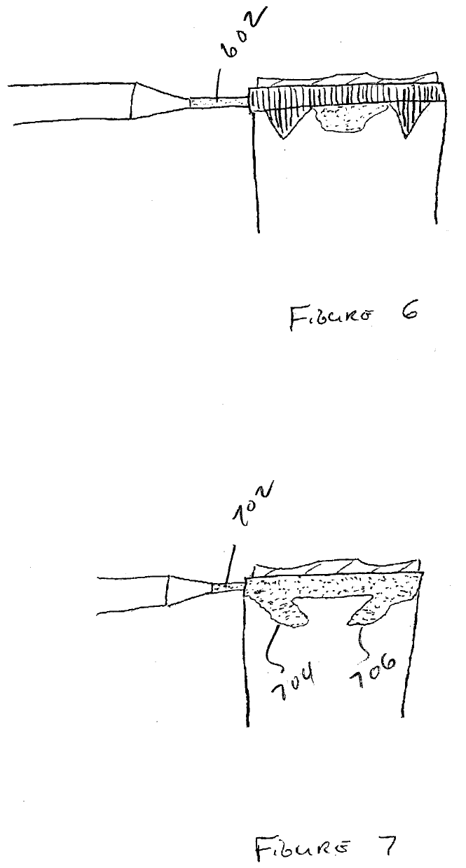

FIG. 5A is a lateral view of an alternative embodiment of the invention, wherein an in situ hardening “liquid metal” or other polymer 502, is injected into a mold or cavity in the disc space. FIG. 5B is a sagittal cross section of another embodiment of the invention, wherein “liquid metal” or a polymer is injected to improve the fit of a standard ADR EP. The material can be injected through a hole 550 in the vertebra 560. Alternatively, the material can be injected through a hole in the ADR EP.

The invention is useful for other areas of the body, including hips, knees, shoulders, and elbows. FIG. 6 is a coronal cross section of the tibia and an embodiment of the invention for prosthetic knees. The material 602 is injected to improve the fit between the tibial tray and the tibia.

FIG. 7 is a coronal cross section of the tibia and another embodiment of the invention. The material 702 hardens in diverging or converging holes 704, 706 to improve the pull-out strength of the tibial tray.

I claim:

1. An anatomical artificial disc replacement (ADR) device, comprising:
a tray having a surface which is convex to better conform to a concavity in a vertebral endplate.

2. The ADR device of claim 1, wherein the tray is constructed of multiple pieces adapted to conform to the vertebral endplate.

3. The ADR device of claim 1, wherein the tray is constructed of a flexible material to fit the vertebral endplate.

4. The ADR device of claim 3, wherein the flexible material is a malleable metal.

5. The ADR device of claim 1, wherein the tray includes a substrate and an attachable convex piece configured to conform to the concavity.

6. The ADR device of claim 1, wherein the tray includes a substrate and an injectable material that hardens in situ to fill the concavity.

7. The ADR device of claim 6, wherein the injectable material is a liquid metal.

8. The ADR device of claim 6, wherein the injectable material is a polymer.

9. The ADR device of claim 6, wherein the material is injected along diverging or converging paths to minimize pull-out.
